(19) United States
(12) Patent Application Publication
(10) Pub. No.: US 2023/0411020 A1
(43) Pub. Date: Dec. 21, 2023

(54) METHODS FOR QUALITY OF LIFE ASSESSMENT IN COMPANION ANIMALS

(71) Applicant: MARS, INCORPORATED, McLean, VA (US)

(72) Inventor: Geert DE MEYER, Melton Mowbray (GB)

(73) Assignee: MARS, INCORPORATED, McLean, VA (US)

(21) Appl. No.: 18/336,125

(22) Filed: Jun. 16, 2023

Related U.S. Application Data
(60) Provisional application No. 63/353,100, filed on Jun. 17, 2022.

(51) Int. Cl. G16H 50/30; G16H 50/70

(52) U.S. Cl. CPC G16H 50/30; G16H 50/70

(57) ABSTRACT

Methods of characterizing health states and overall Quality of life (QoL) for animals are provided. Predictive modeling and computer systems related to health assessments are also provided.

### PRIORITY

This application claims the benefit under 35 U.S.C. § 119(e) of U.S. Provisional Patent Application No. 63/353,100, filed 17 Jun. 2022, which is incorporated herein by reference in its entirety.

### FIELD

The presently disclosed subject matter relates to methods of assessing quality of life in companion animals through novel instrumentation. The disclosed subject matter provides the ability to quantify well-being through targeted domains on a large scale.

### BACKGROUND

Quality-of-Life (QoL) is a multidimensional concept of an individual's evaluation of aspects of her/his/their life as diverse as physical, social, and emotional well-being. [1] In practice, QoL is quantified by survey-based instruments that deliver a set of QoL domain scores, one for each QoL aspect considered. QoL domain scores are either obtained directly by scoring QoL domains in a small number of categories [2, 3] or indirectly through mapping multiple survey item scores on domain constructs by means of psychometric test methodologies. [4] Collectively, the QoL domain scores are called the health state. It is commonly accepted that the health state fully describes an individual's QoL despite that the link between the set of domains and overall QoL is primarily based on qualitative grounds. [5]

In general, QoL instruments also collect an overall QoL score that is typically measured on a 0-100 visual analog scale (VAS), and is therefore called the VAS score. This VAS score can be reliably used within a specific context (e.g., a cancer trial) but not across different contexts (e.g., comparing cancer and dementia patients) because it is prone to context-specific bias. To mitigate this limitation, a utility index score was developed as an alternative overall QoL metric. The utility index score is computed from a categorical health state (a health state with a small number of categories for each domain) by a set of mappings (value set) that convert every health state instance to a utility index score. The value set is estimated in a valuation experiment in which a randomly selected group of 500-1000 people scores sets of health state instances for overall QoL using either a time trade-off method, a standard gamble method, or a visual analog method. [6] As such, the value set represents a general population-level appreciation of differences between health state instances rather than an affected individual's perception of her/his own health state instance. This gives the utility score general validity which has made it the preferred overall QoL metric for health economic studies. Human VAS and utility scores distributions are well characterized by location (e.g., at country level) and, for both, metrics age-specific reference ranges are generally available.

Over the last 5 years, several QoL instruments have been developed to quantify QoL of dogs and cats with the owner filling in the survey as a proxy. [7, 8, 9, 10] However, this methodology was limited in that the assessments were exclusively focused on the QoL domain scores, however, did not focus on overall QoL. Thus, there remains a need in the art for a comprehensive system and methodology to address overall QoL distribution in general populations of companion animals.

### SUMMARY

The purpose and advantages of the disclosed subject matter will be set forth in and are apparent from the description that follows, as well as will be learned by practice of the disclosed subject matter. Additional advantages of the disclosed subject matter will be realized and attained by the devices particularly pointed out in the written description and claims thereof, as well as from the appended drawings. To achieve these and other advantages and in accordance with the purpose of the disclosed subject matter, as embodied and broadly described, the disclosed subject matter includes methods of characterizing and measuring a Categorical Health State for companion animals.

In one embodiment, the presently disclosed subject provides a method implemented by a computer system, comprising: receiving data associated with health and wellness of an animal, wherein the data comprises at least a Visual Analog Scale (VAS) score, and domain scores associated with each of a plurality of indicators; determining a correlation between the VAS score and the domain scores associated with each of the plurality of indicators; and determining a total quality of life (QoL) score based at least in part on the correlation.

In certain embodiments, the method further comprises determining, based on the correlation between the VAS score and the domain scores associated with each of the plurality of indicators, a subset of the plurality of indicators; and determining the total QoL score based on correlations between the VAS score and domain scores associated with the subset of the plurality of indicators.

In certain embodiments, the method further comprises classifying the domain scores associated with each of a plurality of indicators into more than one category.

In certain embodiments, the VAS scores are scalable. In certain embodiments, the domain scores are scalable.

In certain embodiments, determining the total QoL score is further based on animal breed, animal size, and animal gender,

In certain embodiments, the method further comprises determining, using a machine-learning model, the total QoL score based at least in part on the VAS score, the domain scores associated with each of the plurality of indicators, and the correlation.

In certain embodiments, the indicators comprise one or more of daytime energy, daytime relaxation, daytime sociability, daytime mobility, daytime happiness, mealtime relaxation, mealtime interest, or mealtime satisfaction.

The presently disclosed subject matter also provides a system comprising one or more non-transitory computer-readable storage media including instructions; and one or more processors coupled to the storage media, the one or more processors configured to execute the instructions to: receive data associated with health and wellness of a pet, wherein the data comprises at least a Visual Analog Scale (VAS) score, and domain scores associated with each of a plurality of indicators; determine a correlation between the VAS score and the domain scores associated with each of the plurality of indicators; and determine a total QoL score based at least in part on the correlation.

The presently disclosed subject matter also provides a non-transitory computer-readable medium comprising instructions that, when executed by one or more processors of a computing system, cause the one or more processors to: receive data associated with health and wellness of a pet, wherein the data comprises at least a Visual Analog Scale (VAS) score, and domain scores associated with each of a plurality of indicators; determine a correlation between the VAS score and the domain scores associated with each of the plurality of indicators; and determine total QoL score based at least in part on the correlation.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, without departing from the scope of this disclosure.

FIGS. 1A-1H illustrate a boxplot representation of the QoL domain score distribution as a function of VAS score. Spearman's rank correlation estimates are shown in the panel header. FIG. 1A is directed to the Day Energetic domain. FIG. 1B is directed to the Day Relaxed domain. FIG. 1C is directed to the Day Sociable domain. FIG. 1D is directed to the Day Mobile domain. FIG. 1E is directed to the Day Happy domain. FIG. 1F is directed to the Meal Relaxed domain. FIG. 1G is directed to the Meal Interested domain. FIG. 1H is directed to the Meal Satisfied domain.

FIGS. 2A-2F illustrate a frequency distribution of VAS scores as a function of binned QoL domain scores. Spearman' s rank correlation estimates are shown in the panel header. FIG. 2A is directed to the Day Energetic domain. FIG. 2B is directed to the Day Relaxed domain. FIG. 2C is directed to the Day Sociable domain. FIG. 2D is directed to the Day Mobile domain. FIG. 2E is directed to the Day Happy domain. FIG. 2F is directed to the Meal Interested domain.

FIGS. 3A and 3B illustrate a model fit for the 5-domain main effects model (model 2, FIG. 3A) and a 5-domain model with 2-way interactions (model 4, FIG. 3B) for 79 health states instances with 5 or more observations and bubble size reflecting the number of observations per instance.

FIG. 4 illustrates an example computer system of the disclosed subject matter.

### DETAILED DESCRIPTION

The present disclosure is based, in part, on the discovery that overall QoL for companion animals can be quantified based on the development of various methods including VAS and Utility Index Scores. Non-limiting embodiments of the invention are described by the present specification and Examples.

For clarity, but not by way of limitation, the detailed description of the presently disclosed subject matter is divided into the following subsections:
I. Definitions;
II. Companion Animals; and
III. Data Modeling.

### I. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

As used herein, the use of the word “a” or “an” when used in conjunction with the term “comprising” in the claims and/or the specification can mean “one,” but it is also consistent with the meaning of “one or more,” “at least one,” and “one or more than one.”

The terms “comprise(s),” “include(s),” “having,” “has,” “can,” “contain(s),” and variants thereof, as used herein, are intended to be open-ended transitional phrases, terms or words that do not preclude additional acts or structures. The present disclosure also contemplates other embodiments “comprising,” “consisting of” and “consisting essentially of,” the embodiments or elements presented herein, whether explicitly set forth or not.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value.

The terms “animal” or “pet” as used in accordance with the present disclosure refers to domestic animals including, but not limited to, domestic dogs, domestic cats, horses, cows, ferrets, rabbits, pigs, rats, mice, gerbils, hamsters, goats, and the like. Domestic dogs and cats are particular non-limiting examples of pets. The term “animal” or “pet” as used in accordance with the present disclosure can further refer to wild animals, including, but not limited to bison, elk, deer, duck, fowl, fish, and the like.

As used herein, the term “companion animal” refers to animals such as a cat, a dog, a guinea pig, a rabbit, a bird, or a horse. For example, but not by way of limitation, the companion animal can be a “domestic” dog, e.g., Canis lupus familiaris. In certain embodiments, the companion animal can be a “domestic” cat such as Felis domesticus.

As used herein, the term “spay” or “spaying” refers to the complete removal of the ovaries and uterus in order to sterilize a female animal.

As used herein, the term “neuter” or “neutering” refers to the removal of the testicles in order to sterilize a male animal.

### II. Companion Animals

The presently disclosed subject matter focuses on the health assessment of companion animals. In specific embodiments, the companion animal is a domestic dog.

### Dog Breeds

The present disclosure relates to, inter alfa, methods for assessing health and well-being of animals. Characteristics of companion animals, including size, sex, and breed, can vary between species. However, for the most common member within this category, dogs, can in general provide an indication of the efficacy of a method when applied to other animals.

As used herein, the expression “size category” refers to the definition of the animal (e.g., dogs, cats, etc.) in terms of the average weight of the particular animal breed. Animals (e.g., dogs, cats, etc.) of the same breed can have relatively uniform physical characteristics, such as size, coat color, physiology, and behavior, as compared to animals of a different breed. It is noted that the discussion below is focused on dogs, however, other companion animals and wild animals are intended to be covered by the scope of this disclosure and the present disclosure is not intended to be limited to dogs.

The dog can be any breed of dog, including toy, small, medium, large or giant breeds. Non-limiting examples of toy breeds include Affenpinscher, Australian Silky Terrier, Bichon Frise, Bolognese, Cavalier King Charles Spaniel, Chihuahua, Chinese Crested, Coton De Tulear, English Toy Terrier, Griffon Bruxellois, Havanese, Italian Greyhound, Japanese Chin, King Charles Spaniel, Lowchen (Little Lion Dog), Maltese, Miniature Pinscher, Papillon, Pekingese, Pomeranian, Pug, Russian Toy, and Yorkshire Terrier. Examples of small breeds include, but are not limited to, French Bulldog, Beagle, Dachshund, Pembroke Welsh Corgi, Miniature Schnauzer, Cavalier King Charles Spaniel, Shih Tzu, and Boston Terrier. Examples of medium dog breeds include, but are not limited to, Bulldog, Cocker Spaniel, Shetland Sheepdog, Border Collie, Basset Hound, Siberian Husky, and Dalmatian. Examples of large breed dogs include, but are not limited to, Great Dane, Neapolitan mastiff, Scottish Deerhound, Dogue de Bordeaux, Newfoundland, English mastiff, Saint Bernard, Leonberger, and Irish Wolfhound. Cross-breeds can generally be categorized as toy, small, medium, and large dogs depending on their body weight. In certain embodiments, the dog is a toy breed. In certain embodiments, the dog is a medium, large or giant breed. In some embodiments, the dog is a mix of two or more breeds. In such instances, the mixed-breed dog can still be categorized by size depending on their body weight and can exhibit traits (e.g., behavioral traits, genetic traits, etc.) associated with each of the two or more breeds found in the dog.

The Fédération Cynologique Internationale currently recognizes 346 pure dog breeds. The breed of a dog can be identified, for example, either by observing its physical traits or by genetic analysis. A pedigree dog is the offspring of two dogs of the same breed, which is eligible for registration with a recognized club or society that maintain a register for dogs of that description. There are a number of pedigree dog registration schemes, of which the Kennel Club is the most well-known. Categories according to the Kennel Club are defined as follows in Table 1.

TABLE 1
A list of dog size categories.

| Size Category name | Weight range of dog breed | |
|---|---|---|
| Toy/Extra small | Up to 6.5 Kg | |
| Small | 6.5-9 | Kg |
| Medium-small | 9-15 | Kg |
| Medium-large | 15-30 | Kg |
| Large | 30-40 | Kg |

| | |
|---|---|
| Giant | Over 40 Kg |

In certain embodiments, the dog size categories are selected according to the Kennel Club. In other embodiments, the dog size categories are selected according to alternative designations. For example, in certain embodiments, the toy and small dog breeds have an average body weight of up to about 10 kg. In certain embodiments, the medium dog breeds have an average body weight of from about 10 kg to about 25 kg. In certain embodiments, the large dog breeds have an average body weight of from at least about 25 kg or more.

### III. Data Modeling

The presently disclosed methods are based on data that includes particular measurement indices or indications derived from but not limited to QoL surveys, Domain categories and scores, as well as overall QoL scores.

In certain embodiments, QoL surveys can include a variety of questions directed to health and/or wellness parameters. In certain embodiments, EQ-5D questionnaires (established by the EuroQol Group) can be used. The EQ-5D system includes five dimensions: mobility, self-care, usual activities, pain and discomfort, and anxiety and depression. In other embodiments, any QoL survey that includes scaled or quantified measurement can be used to obtain appropriate QoL information on a target population.

The surveyed data can cover one or more domains that link a specified environment with different indicators for health and wellness. For example, the environment can be a daytime or mealtime setting. The indicators can include levels of energy, relaxation, sociability, mobility, and happiness. Alternatively, the indicators can include relaxation, interest, and satisfaction.

In certain embodiments, the domains can include daytime energy, daytime relaxation, daytime sociability, daytime mobility, and/or daytime happiness. In certain embodiments, the domains can include mealtime relaxation, mealtime interest, and/or mealtime satisfaction.

The presently disclosed subject matter uses surveyed data to provide “domains scores” of health and wellness as well as overall QoL scores based on the surveys that are administered. In certain embodiments, the presently disclosed subject matter, overall QoL scores are collected on an integer scale from 1 (labelled ‘not good at all’) to 9 (labelled ‘could not be better’) and can be rescaled to a VAS score in the 0 to 100 range for purposes of further modeling discussed below. In certain embodiments, the animal's life stage is computed from age and breed information provided through the surveys or questionnaires. The data points are then utilized to develop a Categorical Health State.

The present disclosure describes solutions to determining overall QoL for companion animals with certain embodiments of systems and methods for predicting the overall QoL based on data collected from the survey items related to the companion animal's physiological characteristics and/or health and wellness of the companion animal. In accordance with some embodiments, the disclosed systems and methods characterizing and measuring overall QoL for companion animals to provide the ability to quantify well-being of the companion animals. To do so, the systems and methods can receive data associated with the health and wellness of the companion animals. The data can be collected from the survey of QoL instruments, and can comprise data related to a group of companion animals that participate the survey. The companion animals can have diverse physiological characteristics, such as gender, size, breed, age, etc. The data can comprise Visual Analog Scale (VAS) scores, each of the VAS scores can relate to each of the companion animals. The survey can comprise multiple indicators relate to the health and wellness of companion animals. The data received can comprise domain scores for each of the indicators for each of the companion animals. For example, a particular companion animal can provide data comprising a VAS score associated with the particular companion animal, and multiple domain scores for the health and wellness indicators, wherein each indicator corresponds to one domain score for the particular animal. In some embodiments, the indicators comprise one or more of daytime energy, daytime relaxation, daytime sociability, daytime mobility, daytime happiness, mealtime relaxation, mealtime interest, or mealtime satisfaction.

Based on the data received for the group of animals, the computer systems and methods can determine, for each indicator, a correlation between the domain score and the VAS score. The correlation can be calculated based on all the VAS scores received and the received domain scores associated with a particular indicator. In some embodiments, the correlation can be calculated based on a portion of the VAS scores received, and a portion of the received domain scores associated with a particular indicator. In some embodiments, the domain scores can be classified into multiple categories. The computer systems and the methods can determine multiple correlations for the multiple indicators. The correlations can comprise one or more of linear relationship and/or not highly related relationship. The computer systems and methods can discard the data associated with not highly related indicators. The computer systems and methods can determine a subset of VAS scores and domains scores associated with selected indicators based on the correlations. For example, based on the correlation that indicates a linear relationship between the VAS scores and the domain scores for the particular indicator, the systems and methods can select the particular indicator and data associated with the particular indicator for subsequent processing.

The computer system and methods can train a machine-learning model using the VAS scores and the domain scores to provide output indicating a total QoL score based on the received domain scores for the companion animal. The computer systems and methods can use a multiple linear regression model to determine a predicted total QoL score based on the scattered VAS scores and domains scores for multiple indicators. The computer systems and method can determine the total QoL score based on:

yi=β0+β1xi1+β2xi2+. . . +βpxip+∈,

wherein the dependent variable yi can be the total QoL score, and explanatory variables xi can be domain scores associated with p indicators. The computer systems and methods can train the model with a ground truth to determine the optimized parameters β. The ground truth can be VAS scores. The systems and methods can use a least-square method to determine the parameters β. In some embodiments, the VAS scores and the domains scores are scalable. The computer systems and methods can determine a total QoL score, using the trained machine model (e.g., multiple linear regression model) to determine well-being of companion animals.

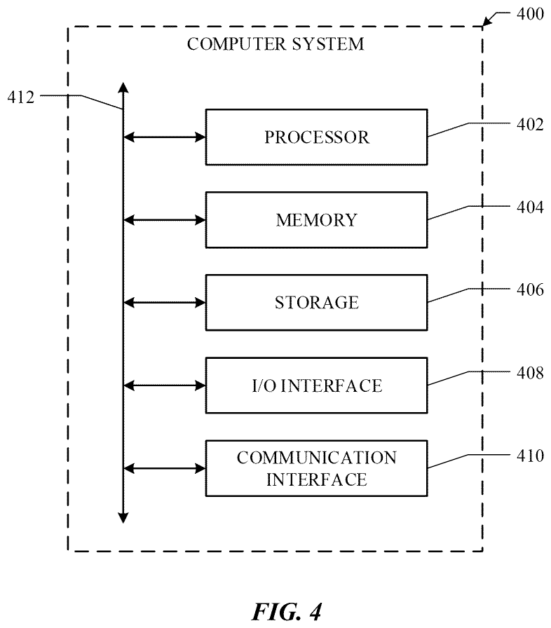

FIG. 4 illustrates an example computer system 400. In particular embodiments, one or more computer systems 400 perform one or more steps of one or more methods described or illustrated herein. In particular embodiments, one or more computer systems 400 provide functionality described or illustrated herein. In particular embodiments, software running on one or more computer systems 400 performs one or more steps of one or more methods described or illustrated herein or provides functionality described or illustrated herein. Particular embodiments include one or more portions of one or more VAS computer systems 400. Herein, reference to a computer system can encompass a computing device, and vice versa, where appropriate. Moreover, reference to a computer system can encompass one or more computer systems, where appropriate.

This disclosure contemplates any suitable number of computer systems 400. This disclosure contemplates computer system 400 taking any suitable physical form. As example and not by way of limitation, computer system 400 can be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 400 can include one or more computer systems 400; be unitary or distributed; span multiple locations; span multiple machines; span multiple data centers; or reside in a cloud, which can include one or more cloud components in one or more networks. Where appropriate, one or more computer systems 400 can perform without substantial spatial or temporal limitation one or more steps of one or more methods described or illustrated herein. As an example and not by way of limitation, one or more computer systems 400 can perform in real time or in batch mode one or more steps of one or more methods described or illustrated herein. One or more computer systems 400 can perform at different times or at different locations one or more steps of one or more methods described or illustrated herein, where appropriate.

In particular embodiments, computer system 400 includes a processor 402, memory 404, storage 406, an input/output (I/O) interface 408, a communication interface 410, and a bus 412. Although this disclosure describes and illustrates a particular computer system having a particular number of particular components in a particular arrangement, this disclosure contemplates any suitable computer system having any suitable number of any suitable components in any suitable arrangement.

In particular embodiments, processor 402 includes hardware for executing instructions, such as those making up a computer program. As an example and not by way of limitation, to execute instructions, processor 402 can retrieve (or fetch) the instructions from an internal register, an internal cache, memory 404, or storage 406; decode and execute them; and then write one or more results to an internal register, an internal cache, memory 404, or storage 406. In particular embodiments, processor 402 can include one or more internal caches for data, instructions, or addresses. This disclosure contemplates processor 402 including any suitable number of any suitable internal caches, where appropriate. As an example and not by way of limitation, processor 402 can include one or more instruction caches, one or more data caches, and one or more translation lookaside buffers (TLBs). Instructions in the instruction caches can be copies of instructions in memory 404 or storage 406, and the instruction caches can speed up retrieval of those instructions by processor 402. Data in the data caches can be copies of data in memory 404 or storage 406 for instructions executing at processor 402 to operate on; the results of previous instructions executed at processor 402 for access by subsequent instructions executing at processor 402 or for writing to memory 404 or storage 406; or other suitable data. The data caches can speed up read or write operations by processor 402. The TLBs can speed up virtual-address translation for processor 402. In particular embodiments, processor 402 can include one or more internal registers for data, instructions, or addresses. This disclosure contemplates processor 402 including any suitable number of any suitable internal registers, where appropriate. Where appropriate, processor 402 can include one or more arithmetic logic units (ALUs); be a multi-core processor; or include one or more processors 402. Although this disclosure describes and illustrates a particular processor, this disclosure contemplates any suitable processor.

In particular embodiments, memory 404 includes main memory for storing instructions for processor 402 to execute or data for processor 402 to operate on. As an example and not by way of limitation, computer system 400 can load instructions from storage 406 or another source (such as, for example, another computer system 400) to memory 404. Processor 402 can then load the instructions from memory 404 to an internal register or internal cache. To execute the instructions, processor 402 can retrieve the instructions from the internal register or internal cache and decode them. During or after execution of the instructions, processor 402 can write one or more results (which can be intermediate or final results) to the internal register or internal cache. Processor 402 can then write one or more of those results to memory 404. In particular embodiments, processor 402 executes only instructions in one or more internal registers or internal caches or in memory 404 (as opposed to storage 406 or elsewhere) and operates only on data in one or more internal registers or internal caches or in memory 404 (as opposed to storage 406 or elsewhere). One or more memory buses (which can each include an address bus and a data bus) can couple processor 402 to memory 404. Bus 412 can include one or more memory buses, as described below. In particular embodiments, one or more memory management units (MMUs) reside between processor 402 and memory 404 and facilitate accesses to memory 404 requested by processor 402. In particular embodiments, memory 404 includes random access memory (RAM). This RAM can be volatile memory, where appropriate. Where appropriate, this RAM can be dynamic RAM (DRAM) or static RAM (SRAM). Moreover, where appropriate, this RAM can be single-ported or multi-ported RAM. This disclosure contemplates any suitable RAM. Memory 404 can include one or more memories 404, where appropriate. Although this disclosure describes and illustrates particular memory, this disclosure contemplates any suitable memory.

In particular embodiments, storage 406 includes mass storage for data or instructions. As an example and not by way of limitation, storage 406 can include a hard disk drive (HDD), a floppy disk drive, flash memory, an optical disc, a magneto-optical disc, magnetic tape, or a Universal Serial Bus (USB) drive or a combination of two or more of these. Storage 406 can include removable or non-removable (or fixed) media, where appropriate. Storage 406 can be internal or external to computer system 400, where appropriate. In particular embodiments, storage 406 is non-volatile, solid-state memory. In particular embodiments, storage 406 includes read-only memory (ROM). Where appropriate, this ROM can be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 406 taking any suitable physical form. Storage 406 can include one or more storage control units facilitating communication between processor 402 and storage 406, where appropriate. Where appropriate, storage 406 can include one or more storages 406. Although this disclosure describes and illustrates particular storage, this disclosure contemplates any suitable storage.

In particular embodiments, I/O interface 408 includes hardware, software, or both, providing one or more interfaces for communication between computer system 400 and one or more I/O devices. Computer system 400 can include one or more of these I/O devices, where appropriate. One or more of these I/O devices can enable communication between a person and computer system 400. As an example and not by way of limitation, an I/O device can include a keyboard, keypad, microphone, monitor, mouse, printer, scanner, speaker, still camera, stylus, tablet, touch screen, trackball, video camera, another suitable I/O device or a combination of two or more of these. An I/O device can include one or more sensors. This disclosure contemplates any suitable I/O devices and any suitable I/O interfaces 408 for them. Where appropriate, I/O interface 408 can include one or more device or software drivers enabling processor 402 to drive one or more of these I/O devices. I/O interface 408 can include one or more I/O interfaces 408, where appropriate. Although this disclosure describes and illustrates a particular I/O interface, this disclosure contemplates any suitable I/O interface.

In particular embodiments, communication interface 410 includes hardware, software, or both providing one or more interfaces for communication (such as, for example, packet-based communication) between computer system 400 and one or more other computer systems 400 or one or more networks. As an example and not by way of limitation, communication interface 410 can include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 410 for it. As an example and not by way of limitation, computer system 400 can communicate with an ad hoc network, a personal area network (PAN), a local area network (LAN), a wide area network (WAN), a metropolitan area network (MAN), or one or more portions of the Internet or a combination of two or more of these. One or more portions of one or more of these networks can be wired or wireless. As an example, computer system 400 can communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 400 can include any suitable communication interface 410 for any of these networks, where appropriate. Communication interface 410 can include one or more communication interfaces 410, where appropriate. Although this disclosure describes and illustrates a particular communication interface, this disclosure contemplates any suitable communication interface.

In particular embodiments, bus 412 includes hardware, software, or both coupling components of computer system 400 to each other. As an example and not by way of limitation, bus 412 can include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 412 can include one or more buses 412, where appropriate. Although this disclosure describes and illustrates a particular bus, this disclosure contemplates any suitable bus or interconnect.

Herein, a computer-readable non-transitory storage medium or media can include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium can be volatile, non-volatile, or a combination of volatile and non-volatile, where appropriate.

### EXAMPLES

The presently disclosed subject matter will be better understood by reference to the following Example, which is provided as exemplary of the presently disclosed subject matter, and not by way of limitation.

### EXAMPLE 1: Characterizing Overall Quality of Life (QoL) in Dogs

The present Example is directed to characterizing and modeling overall QoL in the general dog population based on domain score assessments rescaled to visual analog scores.

### Materials and Methods

Data collection.

This study was based on a total of 2,789 QoL surveys collected using a categorical health state for the dog QoL instrument development work for which 8 QoL domain scores, and an overall QoL score were obtained. The data originated from three different studies that primarily sampled the general dog population in the United States. See Table 2 for basic information from the three studies.

TABLE 2

| Study | Study 1 | Study 2 | Study 3 |
|---|---|---|---|
| Geography | United Kingdom | United States | United States |
| | and United States | | |
| Respondents | 310 | 868 | 1635 |
| (items) | | | |
| Respondents | 310 | 868 | 1611 |
| (reported overall | | | |
| QoL score) | | | |
| Dog age in years | — | 7.3 (3.6) | 6.5 (4.1) |
| (standard deviation) | | | |
| Dog sex | — | 50.4-49.6 | 47.0-53.0 |
| (% female-% male) | | | |

Overall QoL scores were collected on an integer scale from 1 (labelled ‘not good at all’) to 9 (labelled ‘could not be better’) and were rescaled to a VAS score in the 0 to 100 range. For a subset of 2,465 dogs from Study 2 and Study 3, the dog's life stage was computed from available age and breed information as previously described in earlier studies. Study objectives were shared prior to the survey, and participants consented to these by completing the survey. The usage of electronic medical record data for scientific purposes was also consented to.

### Developing a Categorical Health State for the Dog QoL Instrument

A 6-domain structure with 3 categories per domain corresponding to 3⁶ or 729 possible levels was selected as the starting point for a practical categorical health state. To achieve the required data reduction without major loss of information, individual domain Spearman's rank correlations with the VAS score were used as the guiding metric.

First, the number of domains included was reduced from 8 to 6 by selecting the domains with the highest correlation. Then, the appropriate binning of the remaining QoL domain scores was determined by graphical evaluation of correlation patterns between 6-bin categorization of these domain scores (using the 10th, 25th, 50th, 75th and 90th domain-specific percentiles as cut points) and the VAS score. From this, the 2 most appropriate cut-points to define a 3-category variable were identified. The correlation of that variable with the VAS score was then used to validate the result using the Spearman's rank correlation of the original continuous QoL domain scores with the VAS score as a point of reference.

### Modelling the Expected Overall QoL as a Function of Health State.

To explore how overall QoL relates to the categorical health state, a multiple linear regression model was developed. This model has the VAS score as dependent and 6 categorical predictors (one for each QoL domain included in the categorical health state) with the data above the 25th percentile (high QoL data) serving as reference category. Despite being fitted to individual dog data this model describes the expected (population average) QoL score which can be considered as a proxy for a utility score. Consistent with this population average perspective, a model fit is reported using a root mean squared error derived from the model ‘lack-of-fit’ sum of squares term. In essence, this is the weighted sum of the squared differences between each health state instance's average VAS score and the model prediction for that health state instance. Model building to determine the number of domains required was done by a backward elimination. Subsequently the appropriate set of 2-way interactions was determined through backward elimination from a saturated model with all two-way interactions. Hypothesis tests were based on the F statistic and a significance level of 0.05.

### Results

Unless specified explicitly, data analysis was performed using standard methods in the statistical software R version 3.6.3. Confidence intervals (CI) for reported means were based on the standard error and assumed a normal distribution.

### QoL VAS Score and Domain Scores in the General Dog Population

The distribution of owner reported overall QoL VAS scores was consistent across the three studies and heavily skewed towards the high end of the scale. See Table 3 for a summary of the VAS score distribution for the 3 individual studies and in the combined data set.

TABLE 3

| | | Study | | | | | | | |
|---|---|---|---|---|---|---|---|---|---|
| | | Study 1 | | Study 2 | | Study 3 | | Combined | |
| Reported | 0 | 0 | (0.0) | 0 | (0.0) | 5 | (0.3) | 5 | (0.2) |
| overall QoL | 12.5 | 0 | (0.0) | 0 | (0.0) | 4 | (0.2) | 4 | (0.1) |
| score | 25 | 0 | (0.0) | 2 | (0.2) | 13 | (0.8) | 15 | (0.5) |
| | 37.5 | 2 | (0.6) | 6 | (0.7) | 20 | (1.2) | 28 | (1.0) |
| | 50.0 | 2 | (0.6) | 12 | (1.4) | 58 | (3.6) | 72 | (2.6) |
| | 62.5 | 8 | (2.6) | 31 | (3.6) | 79 | (4.9) | 118 | (4.2) |
| | 75.0 | 46 | (14.8) | 136 | (15.7) | 240 | (14.9) | 422 | (15.1) |
| | 87.5 | 113 | (36.5) | 298 | (34.3) | 446 | (27.7) | 857 | (30.7) |
| | 100.0 | 139 | (44.8) | 383 | (44.1) | 746 | (46.3) | 1268 | (45.5) |

As shown in Table 3, the values represent the number of scores in the study, and the frequency in percent is shown between brackets.

More than 75% of the dog owners rated their dog's overall QoL 87.5% or 100%. Certain percentages are rounded to the nearest even integer as appropriate. Less than 5% scored the overall QoL of their dog at 50% or less. The mean VAS score was 88.2 (95% CI 87.6 to 88.7) and decreased with age from 91.2 (95% CI 90.5 to 91.9, n=1213) in the youth life stage to 87.7 (95% CI 86.6 to 88.7, n=739) in the midlife life stage and 80.5 (95% CI 78.7 to 82.2, n=513) in the senior life stage.

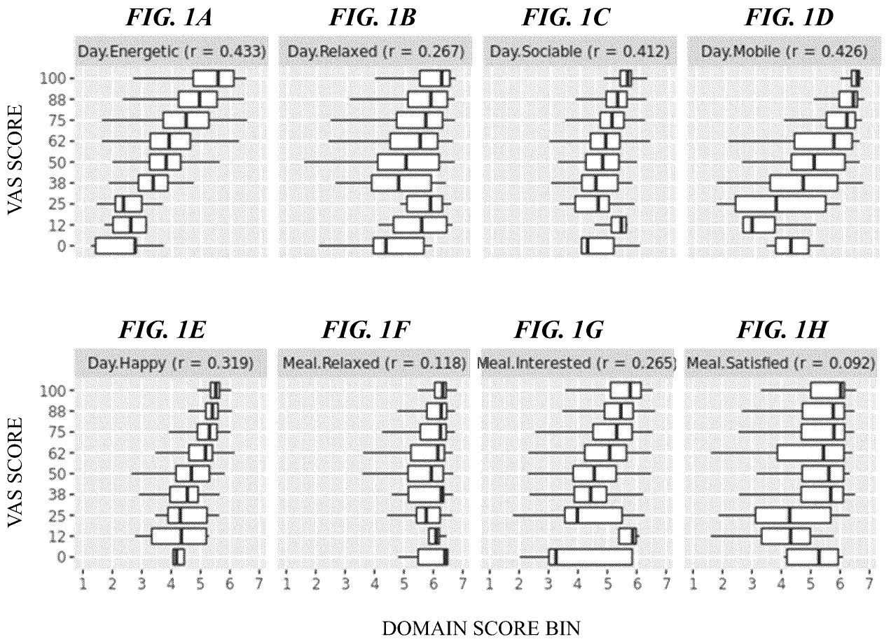

QoL domain scores displayed varying levels of correlation with the VAS score (FIGS. 1A-1H). The relaxed and interested meal domains had a low correlation of about 0.10 while all other domains had a medium correlation ranging from about 0.25 to 0.45. For the medium correlation domains, the correlation is mostly linear particularly when considering that the sparseness in the 0, 13, 25, and 38 VAS score groups makes the boxplot representation less reliable.

### A Categorical Health State for the Dog QoL Instrument

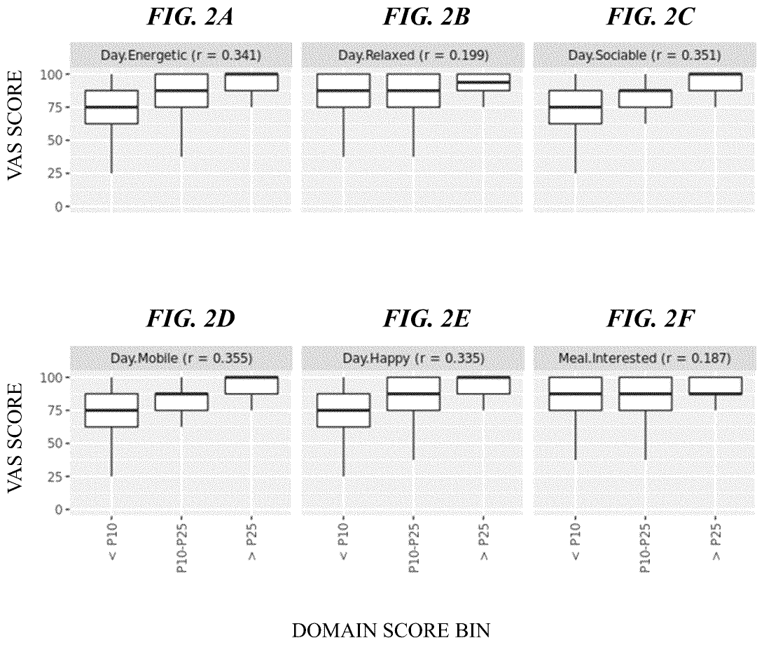

To create a categorical health state that is both informative and dense, a 6 domain 3 category structure was selected as a starting point. The relaxed and interested mealtime domains were excluded from the health state because these showed only a low correlation with the VAS score (FIGS. 1A-1H). Graphical analysis of an initial categorization of the remaining 6 domains using cut points at the 10th, 25th, 50th, 75th and 90th domain-specific percentiles revealed that the correlation with the VAS score was driven by the two low value domain bins (results not shown). Therefore, the 10th and 25th domain-specific percentile cut points were used to create a 3-level categorization for each domain. Spearman's rank correlations between the binned domain scores and the VAS score (FIG. 2) are only slightly smaller than on the original data (FIGS. 1A-1H) which suggests that the binning has retained most of the signal.

### Overall QoL as a Function of Categorical Health State.

A multiple linear regression model for VAS score as a function of the binned QoL domains was developed to characterize the expected (population average) overall QoL in relation to the categorical health state. In a model building strategy starting from a 6-domain health state, the data did not provide evidence for including the relaxed day domain (p=0.2391, model 1 vs. model 2) implying that a 5-domain health state should suffice. See Table 4 for a summary of the model building results for predicting overall QoL from a categorical health state.

TABLE 4

| | | Residual sum | Tested vs | |
|---|---|---|---|---|
| Model | Predictors (number of parameters) | of squares | model | p |
| 01 | Day.Energetic + Day.Relaxed + | 417892 | — | — |
| | Day.Sociable + Day.Mobile + | | | |
| | Day.Happy + Meal.Interested (12) | | | |
| 02 | Day.Energetic + Day.Sociable + | 418323 | 01 | 0.2391 |
| | Day.Mobile + Day.Happy + | | | |
| | Meal.Interested (10) | | | |
| 03 | Day.Energetic + Day.Sociable + | 404232 | 02 | <0.0001 |
| | Day.Mobile + Day.Happy + | | | |
| | Meal.Interested + all two-way | | | |
| | interactions (50) | | | |
| 04 | Day.Energetic + Day.Sociable + | 407098 | 03 | 0.2488 |
| | Day.Mobile + Day.Happy + | | | |
| | Meal.Interested + all two-way | | | |
| | interactions with Day.Energetic + | | | |
| | Day.Sociable*Day.Mobile + | | | |
| | Day.Mobile*Day.Happy (34) | | | |

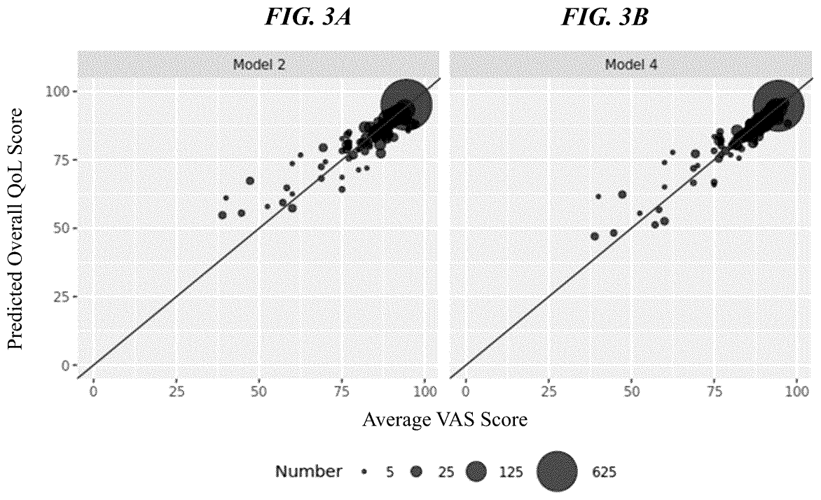

This 5-domain main effects model (model 2) provided accurate results in predicting the expected (population average) overall QoL of a health state. (FIG. 3A). This model has a root mean square error of 5.1 and is primarily driven by the four daytime domains as shown in Table Table 5 provides QoL domain effect estimates for the 5-domain main effect model (model 2). Further, refinement of the 5-domain model showed significant evidence for including interaction terms in general (p<0.0001, model 2 vs model 3) albeit that a subset of all possible 2-way interactions is sufficient (p=0.2488, model 3 vs model 4).

TABLE 5

| Domain | P10-P25 | <P10 | |
|---|---|---|---|
| Day.Energetic | −3.8 (0.7) | −7.0 | (0.9) |
| Day.Sociable | −5.4 (0.7) | −10.0 | (0.7) |
| Day.Mobile | −4.5 (0.7) | −10.8 | (0.9) |
| Day.Happy | −3.8 (0.7) | −10.1 | (0.9) |
| Meal.Interested | −1.8 (0.7) | −2.5 | (0.8) |

Table 5 indicates QoL domain effect estimates for the 5-domain main effect model (model 2) expressed as the reduction in overall QoL score for each domain category starting from the reference category that has an overall QoL score of 95.1. Standard errors are shown between brackets.

The improvement of the 5-domain model with interactions (model 4) over the main effects model (FIG. 3B) leads to a slight reduction of the root mean square error to 4.6 and is mostly apparent in the lower and more accurate predictions for health states with average QoL values in the range between 40 and 60. Likely the interactions terms bring in an additional penalty when multiple QoL domains have lower bin scores. A sample of the expected (population average) overall QoL predictions for a sample of health state instances are shown in Table 6. Table 6 provides a list of number of observations, average VAS score and predicted (population averaged) overall QoL for a set of health state instances.

TABLE 6

| Health | | Average observed | Predicted |
|---|---|---|---|
| State | Number | overall QoL | overall QoL |
| 3 3 3 3 3 | 9 | 38.9 | 47.0 |
| 3 3 3 3 2 | 7 | 44.6 | 48.3 |
| 3 3 3 3 1 | 10 | 60.0 | 52.6 |
| 2 3 1 1 3 | 9 | 76.4 | 75.4 |
| 2 3 1 1 2 | 11 | 69.3 | 77.2 |
| 2 3 1 1 1 | 17 | 81.6 | 80.5 |
| 2 1 1 3 3 | 5 | 80.0 | 80.2 |
| 2 1 1 3 1 | 7 | 76.8 | 85.3 |
| 2 1 1 2 1 | 23 | 87.5 | 88.4 |
| 2 1 1 1 3 | 9 | 87.5 | 86.9 |
| 2 1 1 1 2 | 14 | 89.3 | 88.7 |
| 2 1 1 1 1 | 79 | 93.2 | 91.9 |
| 1 1 1 2 2 | 24 | 94.3 | 89.8 |
| 1 1 1 2 1 | 119 | 91.6 | 90.8 |
| 1 1 1 1 3 | 71 | 93.0 | 93.7 |
| 1 1 1 1 2 | 122 | 94.0 | 93.7 |
| 1 1 1 1 1 | 1054 | 94.5 | 94.7 |

As shown by Table 6, the health states are a coded concatenation of bin identifiers (3 for <P10, 2 for P10-P25 and 1 for >P25) for the Day.Energetic, Day. Sociable, Day.Mobile, Day.Happy and Meal.Interested domains in that order.

### Discussion

This Example has provided the first general population view of overall QoL scores in dogs. In a population of primarily United States dogs, the average VAS score is 88.2 and decreases from 91.2 in young dogs to 80.5 in senior dogs. These scores in the 80 to 90 range are slightly higher than how United States humans (and consequently the dog owners filling out the dog QoL survey) rate their own QoL in the 75 to 85 range. While many factors could account for the observed difference it is quite plausible that age is the major factor. The human estimate is for individuals aged 18 or more while the presently disclosed subject matter has no lower age limit. As a result, a larger proportion of young dogs that have a higher VAS score can lead to the slightly higher VAS scores in the overall dog population. It is also notable that dog QoL VAS scores show a consistent age-related decrease while this likely natural trend is perturbed by psychological factors in humans. In general, it appears though that dog owners rate their dog's overall QoL at very similar levels of magnitude as the human population would rate their own.

For developing a categorical health state, the continuous QoL domain data were summarized into 3 categories with only minor loss of information on the VAS score, as shown FIGS. 2A-2F. Two categories were used to capture the lower end of the QoL domain distribution (values below the 10th percentile and values between the 10th and 25th percentile) while all remaining values were grouped in the third category. This suggests that, when rating overall QoL, dog owners do not attach much importance to the variability above the 25th percentile (degrees of ‘good’) and primarily focus on potential problems expressed in scores below the 25th and 10th percentiles. These findings parallel what has been established for human QoL instruments. For example, both the 5- and 3-category representations of EQ-5D (EuroQol Group) use a top category phrased as ‘I have no problems with (domain denominator)’ and lower categories that capture the extent of problems for that domain. Consequently, potential variation in the ‘no problems’ space is not captured. It thus seems likely that dog owners define good QoL as the absence of problems just like QoL is generally perceived in humans.

To generalize our understanding of overall QoL, the presently disclosed subject matter developed a prediction model for the population-level average VAS score as a function of health state. This model achieved a root mean squared error accuracy of 4.6 with a 5-domain health state consisting of the energetic, sociable, mobile, and happy daytime domains and the interested meal domain (FIG. 3B, Model 4). The four daytime domains had larger effect estimates than the interested mealtime domain (Table 5).

Although possible correlations between predictors preclude causal interpretation and translating effect estimates into relative importance, it is plausible that the energetic, sociable, mobile, and happy daytime domains are the main contributors to overall QoL. It is unsurprising that two ‘physical’ (energetic and mobile) domains and an ‘emotional’ (happy) domain contribute to the overall QoL as these have also been selected on qualitative grounds in another dog QoL instrument. However, it is surprising that the second ‘emotional’ relaxed daytime domain is not included while the sociable daytime domain is. This can be attributed to the sociable domain being intrinsically more important, but might also be a bias of the owner overvaluing the sociable domain through projection. That the relaxed and satisfied mealtime domains are not essential for modelling the population-level QoL VAS score despite displaying significant variation likely implies that dog owners do not link these aspects of the meal as important aspects of the dog's QoL. The small but statistically significant contribution of the interested mealtime domain might be because this domain is seen as indicative of health by the owner. At a more mechanistic level, the domains included mainly operate through additive main affects as evidence by the reasonable fit of model 2 (FIG. 3A) with an almost linear effect within the domain i.e., the effect of the ‘<P10’ category having about twice the size of the ‘P10-P25’ category. On top of these additive effects the best fitting model 4 (FIG. 3B) includes interaction terms.

This implies that problems in multiple domains do not simply add up but get an additional penalty in overall QoL. Taken together, in the presently disclosed subject matter, the modelling of population-level average QoL based on a 5-domain health state is not only feasible, but also grounded on existing understanding of dog health and wellbeing.

At a superficial level, the predicted (population averaged) QoL score can be considered as a proxy for a QoL utility index score. It follows that the predictions derived from model 4 (Table 6) could be used as a value set to compute a utility index score from the 5-domain health state. This interpretation is supported by the fact that modelling the population-level average VAS score smooths the data for the different factors that cause suboptimal QoL and for the variation in appreciation different dog owners might have. Yet, there are also two major benefits from deriving utility index scores from a separate valuation experiment. First is that, in this study, each pet owner only evaluated a single health state while in a valuation study every participant rates a series of health states in a relative sense. Consequently, the method presented in the disclosed subject matter here intrinsically confounds differences between health states with differences in appreciation between pet owners. Given that the relatively parsimonious prediction model (FIG. 3B, model 4) fits the data well, it is unlikely this theoretical limitation has an important practical impact. The second, and likely more important benefit is that it can address the sparseness of data for the low QoL health state instances. The current model fits well in general (as evidenced by the root mean squared error of 4.6) but it likely fits less well for low QoL health state instances. Taken together, the developed overall QoL predictions (Table 6) cannot be used as a value set to compute a QoL utility score. The data in the presently disclosed subject matter do suggest that a carefully designed valuation experiment based on a 5 domain 3 category health state derived from the dog QoL instrument will succeed in developing a dog QoL utility index score.

VAS and future utility index scores for overall QoL in dogs have many potential applications veterinary science. In terms of research, these metrics and associated reference ranges can bring consistency and comparability within and between fields of research, providing objectivity and perspective. For studies into human-animal interaction, if offers an opportunity to collect QoL data from the dog as well as from the human. With proper design strategies, this can distinguish between ‘owner perception’ and ‘intrinsic’ dog QoL components. Furthermore, paired longitudinal data on dog and human QoL has the potential to uncover their causal interplay. The availability of utility index scores for overall dog QoL can also unlock a wide range of health economics strategies that contribute to affordable quality care through optimal use of resources, for example through better pet insurances or hospital loyalty programs. Given these opportunities for veterinary health, the development and deployment of VAS and future utility index scores is a necessary advancement for the art.

### REFERENCES

[1] Quality of Life (2016). York Health Economics Consortium. (yhec.co.uk/glossary/quality-of-life!).
[2] Brazier, J., et al. (2002) The Estimation of a Preference-Based Measure of Health from the SF-36. J Health Econ 21: 271-292.
[3] Herdman, M., et al.(2011) Development and preliminary testing of the new five-level version of EQ-5D (EQ-5D-5L). Qual Life Res 20: 1727-1736.
[4] Ware, J. E. (1999). SF-36 Health Survey. In M. E. Maruish (Ed.), The use of psychological testing for treatment planning and outcomes assessment (pp. 1227-1246). Lawrence Erlbaum Associates Publishers.
[5] U.S. Department of Health and Human Services Food and Drug Administration (2009) Patient-Reported Outcome Measures: Use in Medical Product Development to Support Labeling Claims. (www.fda.gov/media/77832/download).
[6] Whitehead, S. J. & Ali, S. (2010) Health outcomes in economic evaluation: the QALY and utilities. Br Med Bull 96: 5-21.
[7] Freeman, L. M., et al. (2016) Development and initial validation of the Cat HEalth and Wellbeing (CHEW) Questionnaire: a generic health-related quality of life instrument for cats. J Feline Med Surg 18:689-701.
[8] Reid, J., et al. (2018) Shortening of an existing generic online health-related quality of life instrument for dogs. J Small Anim Pract 59:334-342.
[9] Noble, C. E., et al. (2019) Development, initial validation and reliability testing of a web-based, generic feline health-related quality-of-life instrument. J Feline Med Surg 21:84-94.
[10] Schmutz, A., et al. (2022). Development and initial validation of a dog quality of life instrument. Nature Scientific Reports manuscript submitted.
[11] R Core Team. R: A language and environment for statistical computing. (2020).
[12] Jiang, R., et al. (2021) US population norms for the EQ-5D-5L and comparison of norms from face-to-face and online samples. Qual Life Res 30:803-816.
   [13] euroqol.org/eq-5d-instruments.
[14] Spofford, N., et al. (2013). Should the veterinary profession invest in developing methods to assess quality of life in healthy dogs and cats? JAVMA. 243:952-956.

Although the presently disclosed subject matter and its advantages have been described in detail, it should be understood that various changes, substitutions and alterations can be made herein without departing from the spirit and scope of the disclosed subject matter. Moreover, the scope of the present application is not intended to be limited to the particular embodiments of the process, machine, manufacture, and composition of matter, methods and processes described in the specification. As one of ordinary skill in the art will readily appreciate from the disclosed subject matter of the presently disclosed subject matter, processes, machines, manufacture, compositions of matter, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, methods, or steps.

Various patents, patent applications, publications, product descriptions, protocols, and sequence accession numbers are cited throughout this application, the inventions of which are incorporated herein by reference in their entireties for all purposes.

What is claimed is:

1. A method implemented by a computer system, comprising:
receiving data associated with health and wellness of an animal, wherein the data comprises at least a Visual Analog Scale (VAS) score, and domain scores associated with each of a plurality of indicators;
determining a correlation between the VAS score and the domain scores associated with each of the plurality of indicators; and
determining a total quality of life (QoL) score based at least in part on the correlation.

2. The method of claim 1, further comprising:
determining, based on the correlation between the VAS score and the domain scores associated with each of the plurality of indicators, a subset of the plurality of indicators; and
determining the total QoL score based on correlations between the VAS score and domain scores associated with the subset of the plurality of indicators.

3. The method of claim 1, further comprising:
classifying the domain scores associated with each of a plurality of indicators into more than one category.

4. The method of claim 1, wherein the VAS scores are scalable.

5. The method of claim 1, wherein the domain scores are scalable.

6. The method of claim 1, wherein the determining the total QoL score is further based on an animal breed, an animal size, and an animal gender.

7. The method of claim 1, further comprising:
determining, using a machine-learning model, the total QoL score based at least in part on the VAS score, the domain scores associated with each of the plurality of indicators, and the correlation.

8. The method of claim 1, wherein the indicators comprise one or more of daytime energy, daytime relaxation, daytime sociability, daytime mobility, daytime happiness, mealtime relaxation, mealtime interest, or mealtime satisfaction.

9. A system comprising: one or more processors; and a non-transitory memory coupled to the processors comprising instructions executable by the processors, the processors operable when executing the instructions to:
receive data associated with health and wellness of a pet, wherein the data comprises at least a Visual Analog Scale (VAS) score, and domain scores associated with each of a plurality of indicators;
determine a correlation between the VAS score and the domain scores associated with each of the plurality of indicators; and
determine a total QoL score based at least in part on the correlation.

10. The system of claim 9, wherein the processors are further operable when executing the instructions to:
determine, based on the correlation between the VAS score and the domain scores associated with each of the plurality of indicators, a subset of the plurality of indicators; and
determine the total QoL score based on correlations between the VAS score and domain scores associated with the subset of the plurality of indicators.

11. The system of claim 9, wherein the processors are further operable when executing the instructions to:
classify the domain scores associated with each of a plurality of indicators into more than one category.

12. The system of claim 9, wherein the VAS scores are scalable.

13. The system of claim 9, wherein the domain scores are scalable.

14. The system of claim 9, wherein the determining the total QoL score is further based on an animal breed, an animal size, and an animal gender.

15. The system of claim 9, wherein the processors are further operable when executing the instructions to:
determine, using a machine-learning model, the total QoL score based at least in part on the VAS score, the domain scores associated with each of the plurality of indicators, and the correlation.

16. The system of claim 9, wherein the indicators comprise one or more of daytime energy, daytime relaxation, daytime sociability, daytime mobility, daytime happiness, mealtime relaxation, mealtime interest, or mealtime satisfaction.

17. One or more computer-readable non-transitory storage media embodying software that is operable when executed to:
receive data associated with health and wellness of a pet, wherein the data comprises at least a Visual Analog Scale (VAS) score, and domain scores associated with each of a plurality of indicators;
determine a correlation between the VAS score and the domain scores associated with each of the plurality of indicators; and
determine total QoL score based at least in part on the correlation.

18. The media of claim 17, wherein the software is further operable when executed to:
determine, based on the correlation between the VAS score and the domain scores associated with each of the plurality of indicators, a subset of the plurality of indicators; and
determine the total QoL score based on correlations between the VAS score and domain scores associated with the subset of the plurality of indicators.

19. The media of claim 17, wherein the software is further operable when executed to:
classify the domain scores associated with each of a plurality of indicators into more than one category.

20. The media of claim 17, wherein the software is further operable when executed to:
determine, using a machine-learning model, the total QoL score based at least in part on the VAS score, the domain scores associated with each of the plurality of indicators, and the correlation.
